(19) United States
(12) Patent Application Publication
(10) Pub. No.: US 2009/0130222 A1
(43) Pub. Date: May 21, 2009

(54) Methods for Predicting Urine Ph

(76) Inventor: Ryan Michael Yamka, Topeka, KS (US)

Correspondence Address: COLGATE-PALMOLIVE COMPANY, 909 RIVER ROAD, PISCATAWAY, NJ, 08855, US

(21) Appl. No.: 11/995,577

(22) PCT Filed: Jul. 12, 2006
(86) PCT No.: PCT/US06/26863

§ 371 Date: Jan. 14, 2008

Related U.S. Application Data
(60) Provisional application No. 60/698,311, filed on Jul. 12, 2005.

(51) Int. Cl. A61K 33/42; G01N 33/68; G06Q 90/00

(52) U.S. Cl. 424/602; 436/106; 705/500; 206/570; 600/300

(57) ABSTRACT

The invention provides methods for predicting urine pH for an animal by determining the amount of selected cations, anions, and sulfur-containing amino acids in a food for consumption by the animal and predicting urine pH using a formula that equates the amount of such cations, anions, and sulfur-containing amino acids to urine pH. Generally, the amounts of all or a subset of the cations sodium, potassium, calcium, and magnesium; the anions are chloride, sulfur, and phosphorus; and the sulfur-containing amino acids methionine and cystine in a food are used to predict animal urine pH when the food is consumed by the animal.

### CROSS-REFERENCE TO RELATED APPLICATIONS

This application claims priority to U.S. Provisional Application Ser. No. 60/698,311 filed Jul. 12, 2005, the disclosure of which is incorporated herein by reference.

### BACKGROUND OF THE INVENTION

1. Field of the Invention

This invention relates generally to methods for predicting urine pH and particularly to methods for predicting urine pH based upon the composition of a food consumed by an animal.

2. Description of the Prior Art

Urolithiasis is the presence of stones and the process of forming stones in the urinary tract, i.e., the kidney, bladder, and/or urethra. Struvite uroliths are stones in the urinary tract comprising the mineral struvite or magnesium ammonium phosphate hexahydrate. Calcium oxalate uroliths are stones in the urinary tract composed of the mineral calcium oxalate. These uroliths or stones are also referred to as calculi.

The formation of stones in the urinary tract is a significant clinical problem for animals, including companion animals such as dogs and cats. Bacterial urinary tract infection is an important predisposing factor for struvite uroliths. The cause of calcium oxalate uroliths remains unknown. Animal urine pH has been shown to be an important determinant in the prevention and treatment of stone formation. A reduction in urine pH has been shown to reduce the incidence of struvite uroliths. However, a decrease in urine pH may increase the risk of calcium oxalate uroliths.

Diets that lower urine pH may be beneficial in preventing recurrence of struvite uroliths, e.g., a magnesium restricted diet. A diet low in protein, magnesium, and phosphorus and high in salt may be useful to dissolve struvite stones. Diets that increase urine pH may be beneficial in preventing recurrence of calcium oxalate uroliths, e.g., a diet containing potassium citrate or similar compounds.

Dietary ingredients that affect urine pH include sulfur containing amino acids and metabolizable cations and anions. Cations such as calcium, magnesium, sodium and potassium, anions such as sulfur, phosphorus and chloride; and sulfur-containing amino acids such as taurine, methionine, and cysteine have been shown to directly affect urine pH in many animals, including cats, swine, cattle, horses, and rats. While these dietary ingredients are known to affect urine pH, there are no known accurate methods for evaluating a particular food and determining how such food will affect urine pH and urolithiasis if it is consumed by an animal. There is, therefore, a need for new methods for predicting urine pH for an animal consuming a particular diet or food and for new methods for preventing or treating urolithiasis.

### SUMMARY OF THE INVENTION

It is, therefore, an object of the present invention to provide methods and devices for predicting urine pH.

It is another object of the invention to provide methods for preventing or treating urolithiasis.

It is another object of the invention to provide articles of manufacture in the form of kits that contain combinations of devices useful for predicting urine pH and for preventing and treating urolithiasis.

These and other objects are achieved using novel methods for predicting urine pH by determining the amount of selected cations, anions, and sulfur-containing amino acids in a food for consumption by the animal and predicting urine pH using a formula that equates the amount of such cations, anions, and sulfur-containing amino acids to urine pH. Devices useful for predicting urine pH, kits comprising devices useful for predicting urine pH, and various means for communicating information about or instructions for using the present invention are also provided.

Other and further objects, features, and advantages of the present invention will be readily apparent to those skilled in the art.

### DETAILED DESCRIPTION OF THE INVENTION

### Definitions

The term “animal” means a human or other animal susceptible to or suffering from urolithiasis, including avian, bovine, canine, equine, feline, hicrine, murine, ovine, and porcine animals. Preferably, the animal is a canine or feline.

The term “urolithiasis agent” means any drug, food, or other compound or composition useful for preventing or treating urolithiasis, including compositions that alter animal urine pH when consumed by the animal, drugs that lower calcium in the blood, antibiotics, and anti-bacterials.

The symbol “*” in the formulas herein means that the elements on each side of the “*” are multiplied, e.g., (1.2*sodium) means that the amount of sodium in the food is multiplied by 1.2.

The term “in conjunction” means that one or more of the foods, compositions, or compounds (e.g., urolithiasis agents) of the present invention are administered to an animal (1) together in a food composition or (2) separately at the same or different frequency using the same or different administration routes at about the same time or periodically. “Periodically” means that the compositions, food compositions, and compounds are administered on a dosage schedule acceptable for a specific composition, food composition, and compound and that the food compositions are administered or fed to an animal routinely as appropriate for the particular animal. “About the same time” generally means that the compositions, composition components, urolithiasis agents, and food compositions are administered at the same time or within about 72 hours of each other. In conjunction specifically includes administration schemes wherein urolithiasis agents are administered for a prescribed period and the compositions are administered indefinitely.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a method” or “a food composition” includes a plurality of such methods or compositions. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compounds, processes, techniques, procedures, technology, articles, and other compositions and methods disclosed therein that might be used with the present invention. However, nothing herein is to be construed as an admission that the invention is not entitled to antedate such disclosure by virtue of prior invention.

Unless specified otherwise, the amounts of cations, anions, and sulfur-containing amino acids are measured in percent (%) on a dry matter (DM) basis.

### The Invention

In one aspect, the present invention provides a method for predicting urine pH for an animal comprising determining the amount of selected cations, anions, and sulfur-containing amino acids in a food for consumption by the animal and predicting urine pH using a formula that equates the amount of such cations, anions, and sulfur-containing amino acids to urine pH. The invention is based upon the novel discovery that the amounts of certain cations, anions, and sulfur-containing amino acids in a food can be used to predict urine pH and the discovery of the formula or algorithm that equates such amounts to urine pH. The invention is useful for reducing the number of in vitro and in vivo studies required to develop animal foods that will not adversely affect animal urine pH when consumed.

In one embodiment, the invention provides a method for predicting urine pH for an animal comprising determining the amount of the cations sodium, potassium, calcium, and magnesium; the anions chloride, sulfur, and phosphorus; and the sulfur-containing amino acids methionine and cystine in a food for consumption by the animal and predicting urine pH using the formula (Formula 1):

Urine pH=AA+(AB*sodium)+(AC*potassium)−(AD*chloride)−(AE*sulfur)+(AF*calcium)+(AG*magnesium)−(AH*phosphorus)−(AI*methionine)−(AJ*cystine),
where AA is from about 5.5 to about 7.5; AB is from about 1.0 to about 1.3; AC is from about 0.6 to about 0.9; AD is from about 0.6 to about 0.9; AE is from about 0.4 to about 0.9; AF is from about 0.8 to about 0.3; AG is from about 1.0 to about 1.5; AH is from about 0.5 to about 1.0; AI is from about 0.1 to about 0.5; and AJ is from about 0.1 to about 0.5.

In one embodiment, the formula is urine pH=7.05+(1.15*sodium)+(0.72*potassium)−(0.75*chloride)−(0.46*sulfur)+(0.12*calcium)+(1.28*magnesium)−(0.65*phosphorus)−(0.22*methionine)−(0.27*cystine). In another, the formula is urine pH=6.99+(1.29*sodium)+(0.78*potassium)−(0.81*chloride)−(0.49*sulfur)+(0.12*calcium)+(1.22*magnesium)−(0.60*phosphorus)−(0.22*methionine)−(0.27*cystine).

In another embodiment, the invention provides a method for predicting urine pH for an animal comprising determining the amount of the cations sodium, potassium, and magnesium; the anions chloride, sulfur, and phosphorus; and the sulfur-containing amino acids methionine and cystine in a wet food for consumption by the animal and predicting urine pH using the formula (Formula 2):

Urine pH=WA+(WB*sodium)+(WC*potassium)−(WD*chloride)−(WE*sulfur)+(WG*magnesium)−(WH*phosphorus)−(WI*methionine)−(WJ*cystine),
where WA is from about 5.5 to about 7.5; WB is from about 1.2 to about 1.5; WC is from about 0.8 to about 1.2; WD is from about 1.0 to about 1.3; WE is from about 0.1 to about 0.9; WG is from about 3.5 to about 5.5; WH is from about 0.7 to about 1.3; WI is from about 0.3 to about 0.7; and WJ is from about 0.3 to about 0.8.

In one embodiment, the formula is urine pH=7.02+(1.38*sodium)+(0.99*potassium)−(1.12*chloride)−(0.29*sulfur)+(4.51*magnesium)−(0.99*phosphorus)−(0.45*methionine)+(0.50*cystine). In another, the formula is urine pH=7.03+(1.43*sodium)+(0.93*potassium)−(1.16*chloride)−(0.30*sulfur)+(4.76*magnesium)−(0.92*phosphorus)−(0.41*methionine)+(0.34*cystine).

In a further embodiment, the invention provides a method for predicting urine pH for an animal comprising determining the amount of the cations sodium, potassium, calcium, and magnesium; and the anions chloride, sulfur, and phosphorus in a dry food for consumption by the animal and predicting urine pH using the formula (Formula 3):

Urine pH=DA+(DB*sodium)+(DC*potassium)−(DD*chloride)−(DE*sulfur)+(DF*calcium)+(DG*magnesium)−(DH*phosphorus),
where DA is from about 5.5 to about 7.5; DB is from about 1.0 to about 1.3; DC is from about 0.6 to about 0.9; DD is from about 0.6 to about 0.9; DE is from about 0.4 to about 0.6; DF is from about 0.8 to about 0.3; DG is from about 1.0 to about 1.5; and DH is from about 0.5 to about 1.0.

In one embodiment, the formula is urine pH=7.10+(1.03*sodium)+(0.98*potassium)−(0.83*chloride)−(1.70*sulfur)+(0.85*calcium)+(2.07*magnesium)−(1.15*phosphorus). In another, the formula is urine pH=7.03+(1.00*sodium)+(1.00*potassium)−(0.93*chloride)−(1.61*sulfur)+(0.89*calcium)+(1.58*magnesium)−(1.04*phosphorus).

Methods for determining nutrient amounts, metabolite amounts, and urine pH are well known to skilled artisans.

In another aspect, the present invention provides a device useful for predicting urine pH for an animal. The device comprises a means for predicting urine pH that uses a method comprising determining the amount of selected cations, anions, and sulfur-containing amino acids in a food for consumption by the animal and predicting urine pH using a formula that equates the amount of such cations, anions, and sulfur-containing amino acids to urine pH. The means can be any suitable means for performing routine calculations such as a prewritten document, website, software program, calculator, or computer that is designed or preprogrammed to predict urine pH given the amount of selected cations, anions, and sulfur-containing amino acids in a food for consumption by the animal and a formula of the present invention. In certain embodiments, the device accepts input from a user comprising the amount of sodium, potassium, calcium, magnesium, chloride, sulfur, phosphorus, methionine, and cystine in a food for consumption by an animal and utilizes all or a subset of the input and one or more of Formula 1, Formula 2, and Formula 3 to predict urine pH in the animal that consumes the food. In one embodiment, the device is a software program and/or digital media containing such software program designed to permit a user to input data about the cations, anions, and sulfur-containing amino acids in a food and calculate the predicted urine pH using a formula of the present invention. In other embodiments, the device is a computer, calculator, website, or similar device, particularly one that incorporates or utilizes the software program.

In another aspect, the present invention provides a method for preventing or treating urolithiasis. The method comprises determining a desirable urine pH range for an animal; choosing a food containing selected cations, anions, and sulfur-containing amino acids; predicting urine pH using a formula that equates the amount of such cations, anions, and sulfur-containing amino acids in the food to urine pH in the animal that consumes the food; and feeding an urolithiasis preventing or treating amount of the food to the animal if the predicted urine pH is in the desirable pH range.

In one embodiment, (1) the desirable pH range is from about 5.5 to 6.5, (2) the cations, anions, and sulfur-containing amino acids are all or a subset according to the methods of the present invention of the cations sodium, potassium, calcium, and magnesium; the anions chloride, sulfur, and phosphorus, and the sulfur-containing amino acids methionine and cystine, and (3) the formula is Formula 1, Formula 2, or Formula 3 as appropriate for the chosen food and the selected cations, anions, and sulfur-containing amino acids. In one embodiment, the method further comprises feeding the food in conjunction with one or more urolithiasis agents.

In a further aspect, the present invention provides a kit useful for predicting urine pH for an animal and/or for preventing or treating urolithiasis comprising in separate containers in a single package or in a virtual package, as appropriate for the kit component, a device of the present invention and one or more of (1) a food suitable for animal consumption, (2) an urolithiasis agent, (3) a urine pH diagnostic device, (4) a means for communicating information about or instructions for using urine pH diagnostic devices, (5) a means for communicating information about or instructions for adjusting or controlling urine pH, (6) a means for communicating information about or instructions for using the methods, devices, and kits of the present invention to predict urine pH, and (7) a means for communicating information about or instructions for preventing or treating urolithiasis. The kit components are typically in a separate package, in or on the package with one of the other kit components, or in a virtual package, as appropriate for the type of kit component. When the kit comprises a virtual package, the kit is limited to the instructions in a virtual environment in combination with one or more of the other physical kit components. In one embodiment, the food suitable for animal consumption comprises cations, anions, and sulfur-containing amino acids in amounts predicted by the present invention to cause urine pH to be in a desirable range.

In another aspect, the present invention provides a means for communicating information about or instructions for (1) using the methods, devices, and kits of the present invention to predict urine pH, (2) using the methods, devices, and kits of the present invention to prevent or treat urolithiasis, and (3) using urine pH diagnostic devices of the present invention. The communicating means comprises a document, digital storage media, optical storage media, audio presentation, or visual display containing the information or instructions. Preferably, the communication is a displayed website or a brochure, product label, package insert, advertisement, or visual display containing such information or instructions. Useful information and instructions include, but are not limited to, contact information for animals or their caregivers to use if they have a question about the invention and its use and how to use the present invention to calculate urine pH and manage urolithiasis. The communication means is useful for instructing an animal or its caregiver on the benefits of using the present invention.

The composition of foods suitable for consumption by an animal is known to skilled artisans. Typical food ingredients include but are not limited to fats, carbohydrates, proteins, fibers, and nutrients such as vitamins, minerals, and trace elements. Skilled artisans can select the amount and type of food ingredients for a typical food based upon the dietary requirements of the animal, e.g., the animal's species, age, size, weight, health, and function.

The fat and carbohydrate food ingredient is obtained from a variety of sources such as animal fat, fish oil, vegetable oil, meat, meat by-products, grains, other animal or plant sources, and mixtures thereof. Grains include wheat, corn, barley, grain sorghum, and rice.

The protein food ingredient is obtained from a variety sources such as plants, animals, or both. Animal protein includes meat, meat by-products, dairy, and eggs. Meats include the flesh from poultry, fish, and animals such as cattle, swine, sheep, goats, and the like. Meat by-products include lungs, kidneys, brain, livers, stomachs, and intestines. The protein food ingredient may also be free amino acids and/or peptides. Preferably, the protein food ingredient comprises meat, a meat by-product, dairy products, or eggs.

The fiber food ingredient is obtained from a variety of sources such as vegetable fiber sources, e.g., cellulose, beet pulp, peanut hulls, and soy fiber.

The nutrients are obtained from a variety of sources known to skilled artisans, e.g., vitamin and mineral supplements and food ingredients. Vitamins and minerals can be included in amounts required to avoid deficiency and maintain health. These amounts are readily available in the art. The National Research Council (NRC) provides recommended amounts of such nutrients for farm animals. See, e.g., Nutrient Requirements of Swine (10th Rev. Ed., Nat'l Academy Press, Wash. D.C., 1998), Nutrient Requirements of Poultry (9th Rev. Ed., Nat'l Academy Press, Wash. D.C., 1994), Nutrient Requirements of Horses (5th Rev. Ed., Nat'l Academy Press, Wash. D.C., 1989). The American Feed Control Officials (AAFCO) provides recommended amounts of such nutrients for dogs and cats. See American Feed Control Officials, Inc., Official publication, pp. 129-137 (2004). Vitamins generally useful as food additives include vitamin A, vitamin B1, vitamin B2, vitamin B6, vitamin B12, vitamin D, biotin, vitamin K, folic acid, inositol, niacin, and pantothenic acid. Minerals and trace elements useful as food additives include calcium, phosphorus, sodium, potassium, magnesium, copper, zinc, chloride, iron, selenium, iodine, and iron.

The food compositions may contain additional ingredients such as vitamins, minerals, fillers, palatability enhancers, binding agents, flavors, stabilizers, emulsifiers, sweeteners, colorants, buffers, salts, coatings, and the like known to skilled artisans. Stabilizers include substances that tend to increase the shelf life of the composition such as preservatives, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Specific amounts for each composition component, food ingredient, and other ingredients will depend on a variety of factors such as the particular components and ingredients included in the composition; the species of animal; the animal's age, body weight, general health, sex, and diet; the animal's consumption rate; the type of urolithiasis being treated; and the like. Therefore, the component and ingredient amounts may vary widely and may deviate from the preferred proportions described herein.

The food compositions may be canned or wet foods known to skilled artisans. Typically, ground animal proteinaceous tissues are mixed with the other ingredients such as fish oils, cereal grains, balancing ingredients, special purpose additives (e.g., vitamin and mineral mixtures, inorganic salts, cellulose and beet pulp, bulking agents, and the like) and water in amounts sufficient for processing. These ingredients are mixed in a vessel suitable for heating while blending the components. Heating of the mixture is effected using any suitable manner, e.g., direct steam injection or using a vessel fitted with a heat exchanger. Following the addition of the last ingredient, the mixture is heated to a temperature of from about 50° F. to about 212° F. Temperatures outside this range are acceptable but may be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. Sterilization is usually accomplished by heating to temperatures of greater than about 230° F. for an appropriate time depending on the temperature used, the composition, and similar factors. The compositions of the present invention can be added to the food compositions before, during, or after preparation.

The food compositions may be dry foods known to skilled artisans. Typically, dry ingredients such as animal protein, plant protein, grains, and the like are ground and mixed together. Moist or liquid ingredients, including fats, oils, animal protein, water, and the like are then added to and mixed with the dry mix. The mixture is then processed into dry food pieces.

The food compositions may be can be in any form useful for feeding the composition to an animal, e.g., kibbles, treats, and toys for animal food. Kibbles are generally formed using an extrusion process in which the mixture of dry and wet ingredients is subjected to mechanical work at a high pressure and temperature and forced through small openings and cut off into kibble by a rotating knife. The wet kibble is then dried and optionally coated with one or more topical coatings such as flavors, fats, oils, powders, and the like. Kibble also can be made from the dough using a baking process, rather than extrusion, wherein the dough is placed into a mold before dry-heat processing. Treats include compositions that are given to an animal to entice the animal to eat during a non-meal time, e.g., dog bones or biscuits for canines. Treats may be nutritional wherein the composition comprises one or more nutrients or and may have a food-like composition. Non-nutritional treats encompass any other treats that are non-toxic. The composition or components are coated onto the treat, incorporated into the treat, or both. Treats of the present invention can be prepared by an extrusion or baking process similar to those used for dry food. Other processes also may be used to either coat the composition on the exterior of existing treat forms or inject the composition into an existing treat form. Toys include chewable toys such as artificial bones and food compositions shaped to resemble natural foods that are appealing to the animal. The food composition of the present invention can comprise the toy or can form a coating on the surface of the toy or on the surface of a component of the toy. The composition can be incorporated partially or fully throughout the toy or both. In one embodiment, the composition is orally accessible by the intended user. There are a wide range of suitable toys known to skilled artisans, e.g., as shown in U.S. Pat. Nos. 5,339,771 and 5,419,283. The present invention encompasses partially consumable toys, e.g., toys comprising plastic components, and fully consumable toys, e.g., various artificial bones and similar foods. Further, the invention encompasses toys for both human and non-human use, particularly toys for companion, farm, and zoo animal use, and more particularly for feline and canine use.

### EXAMPLES

This invention can be further illustrated by the following examples of preferred embodiments, although it will be understood that these examples are included merely for purposes of illustration and are not intended to limit the scope of the invention unless otherwise specifically indicated.

### Example 1

132 foods (82 dry foods and 50 wet foods) were fed to groups of ten adult (mean=8.5 years of age) cats (felines) to determine the urine pH of cats fed each food. The food was fed for a period of seven days and on days 5 to 7 of the test, urine pH was determined from urine collected at 0730 and 1430 hours.

The cats were cared for in accordance with Institutional Animal Care and Use Committee protocols. The cat's primary living space was cleaned twice daily. Throughout the duration of the experiment, cats were exercised daily and human interaction included but was not limited to play (toys), grooming and other human-cat interactions (i.e. petting). Water was available ad libitum throughout the entire experiment.

The range of nutrients for all dry (82) and wet (50) foods are shown in Tables 1 and 2. Each food was formulated in accordance with the Association of American Feed Control Officials 11 nutrient guide for cats and balanced to meet growth and adult maintenance. Each food was fed to a group of ten cats to maintain body weight. Each day food was offered at 6 hour intervals at 0600, 1200, 1800 and 2400 by an automatic feeding system a to ensure that fresh food was available at all times. Excess food was removed daily and orts were weighed and recorded. Food samples were collected for nutrient content analysis.

TABLE 1
Analyzed Nutrients and Observed Urine pH of the 82 Dry Foods
Used in Feline Urine pH Studies

| Nutrient, | | | | Standard |
|---|---|---|---|---|
| 100% DM Basis | Minimum | Maximum | Average | Deviation |
| Sodium | 0.198 | 0.647 | 0.357 | 0.087 |
| Potassium | 0.307 | 1.542 | 0.805 | 0.207 |
| Chloride | 0.476 | 1.321 | 0.841 | 0.173 |
| Sulfur | 0.465 | 1.149 | 0.722 | 0.172 |
| Calcium | 0.585 | 1.269 | 0.897 | 0.148 |
| Magnesium | 0.043 | 0.182 | 0.088 | 0.039 |
| Phosphorus | 0.486 | 1.064 | 0.755 | 0.095 |
| Methionine | 0.493 | 3.546 | 0.931 | 0.401 |
| Cystine | 0.378 | 1.070 | 0.663 | 0.164 |
| Observed Urine pH | 5.79 | 7.12 | 6.40 | 0.32 |

TABLE 2
Analyzed Nutrients and Observed Urine pH of the 50 Wet Foods
Used in Feline Urine pH Studies

| Nutrient, | | | | Standard |
|---|---|---|---|---|
| 100% DM Basis | Minimum | Maximum | Average | Deviation |
| Sodium | 0.267 | 1.227 | 0.394 | 0.170 |
| Potassium | 0.643 | 1.074 | 0.900 | 0.102 |
| Chloride | 0.172 | 2.876 | 0.883 | 0.416 |
| Sulfur | 0.237 | 2.763 | 0.851 | 0.323 |
| Calcium | 0.469 | 1.338 | 0.945 | 0.214 |
| Magnesium | 0.055 | 0.351 | 0.088 | 0.045 |
| Phosphorus | 0.524 | 1.104 | 0.811 | 0.123 |
| Methionine | 0.345 | 2.005 | 1.353 | 0.338 |
| Cystine | 0.251 | 0.866 | 0.408 | 0.112 |
| Observed Urine pH | 5.85 | 6.98 | 6.40 | 0.288 |

Urine samples were collected twice per day during the course of the one week study at 0730 and 1430 on days 5, 6, and 7. Cat litter boxes were removed from cages at 0600 and returned following the 0730 collection, removed at 1100, and returned following the 1430 collection on days 5, 6, and 7. Urine samples were collected from each cat into a numbered cup by manually expressing the cat's bladder. This ensured that the urine pH was consistent with the actual urine pH in the cat's bladder and prevented contamination from feces if collected naturally. Urine pH was measured using a pH meter.

All feed samples were analyzed for moisture (930.15), calcium (968.08), sodium (968.08), potassium (968.08), magnesium (968.08), chloride (969.10), sulfur (923.01), phosphorus (965.17), methionine and cystine (994.12) according to the Association of Official Analytical Chemists.

After the conclusion of all 132 urine pH studies, the nutrients and observed urine pH values were used to challenge previously published models. These data were then plotted. However, these models failed to accurately predict the average urine pH of the foods used in the current study (r2=0.23, 0.23 and 0.10, respectively).

A new model was developed using previously known cations and anions that effect urine pH with the addition of sulfur in cats fed dry and can foods. Individual mean urine pH per cat (1320 individual urine pH means) was then regressed to the nutrient content (100% dry matter basis) of the food consumed.

Stepwise regression was used to determine which cations and anions were of predictive importance. The cations included in the dry and wet model were sodium, calcium, potassium and magnesium, whereas the anions were chloride, sulfur, phosphorus and the amino acids methionine and cystine. The results are shown in Table 3. The analysis resulted in a prediction equation for foods. The new model accounted for 34% variation in individual (n=1320) observed urine pH and 51% of the variation observed in average (n=132) urine pH in the cats fed 132 foods.

TABLE 3
Urine pH Prediction Models Determined by Stepwise Regression for Individual
Cats (n = 1320) Fed 132 Dry and Wet Foods Using the Nutrient Components
of the Food (% Dry Matter Basis)

| Model | Sodium | Potassium | Chloride | Sulfur | Calcium |
|---|---|---|---|---|---|
| 1 | — | — | −0.29 ± 0.04 | — | — |
| 2 | — | — | −0.49 ± 0.04 | — | — |
| 3 | — | — | −0.55 ± 0.04 | −0.46 ± 0.04 | — |
| 4 | — | — | −0.56 ± 0.04 | −0.49 ± 0.04 | — |
| 5 | — | — | −0.56 ± 0.04 | −0.48 ± 0.04 | — |
| 6 | — | — | −0.53 ± 0.04 | −0.40 ± 0.04 | — |
| 7 | — | 0.42 ± 0.07 | −0.47 ± 0.04 | −0.45 ± 0.04 | — |
| 8 | 1.12 ± 0.14 | 0.72 ± 0.08 | −0.75 ± 0.05 | −0.44 ± 0.04 | — |
| 9 | 1.15 ± 0.14 | 0.72 ± 0.08 | −0.75 ± 0.05 | −0.46 ± 0.04 | 0.17 ± 0.07 |

| Model | Magnesium | Phosphorus | Methionine | Cystine | Intercept | R2 |
|---|---|---|---|---|---|---|
| 1 | — | — | — | — | 6.65 ± 0.03 | 0.05 |
| 2 | 3.23 ± 0.26 | — | — | — | 6.54 ± 0.03 | 0.15 |
| 3 | 3.93 ± 0.26 | — | — | — | 6.88 ± 0.04 | 0.23 |
| 4 | 3.95 ± 0.25 | — | — | −0.30 ± 0.05 | 7.08 ± 0.05 | 0.25 |
| 5 | 4.06 ± 0.25 | −0.50 ± 0.08 | — | −0.36 ± 0.05 | 7.49 ± 0.09 | 0.27 |
| 6 | 3.86 ± 0.25 | −0.47 ± 0.08 | −0.13 ± 0.02 | −0.45 ± 0.05 | 7.59 ± 0.09 | 0.29 |
| 7 | 2.77 ± 0.31 | −0.48 ± 0.08 | −0.19 ± 0.03 | −0.43 ± 0.05 | 7.36 ± 0.09 | 0.31 |
| 8 | 1.33 ± 0.35 | −0.53 ± 0.08 | −0.23 ± 0.03 | −0.29 ± 0.05 | 7.09 ± 0.10 | 0.33 |
| 9 | 1.28 ± 0.35 | −0.65 ± 0.11 | −0.22 ± 0.03 | −0.27 ± 0.05 | 7.05 ± 0.10 | 0.34 |

### Example 2

To determine if separating the food types (dry vs. wet) resulted in higher accuracy in urine pH prediction two more models (dry only and wet only) were developed. The cations included in the wet model were sodium, potassium and magnesium, whereas the anions were chloride, sulfur, phosphorus and the sulfur amino acids methionine and cystine. Calcium was excluded from this model. The results are shown in Table 4. The analysis resulted in a prediction equation for wet foods. The new model accounted for 34% variation in individual (n=1320) observed urine pH and 51% of the variation observed in average (n=132) urine pH in the cats fed 132 foods. The new model accounted for 39% variation in individual (n=500) observed urine pH and 60% of the variation observed in average (n=50) urine pH in the cats fed 50 wet foods.

TABLE 4
Urine pH Prediction Models Determined by Stepwise Regression for Individual
Cats (n = 500) Fed 50 Wet Foods Using the Nutrient Components of the
Food (% Dry Matter Basis)

| Model | Sodium | Potassium | Chloride | Sulfur | Calcium |
|---|---|---|---|---|---|
| 1 | — | — | — | — | — |
| 2 | — | — | — | −0.25 ± 0.05 | — |
| 3 | — | — | — | −0.27 ± 0.05 | — |
| 4 | — | — | −0.17 ± 0.04 | −0.31 ± 0.05 | — |
| 5 | — | — | −0.76 ± 0.08 | −0.29 ± 0.05 | — |
| 6 | 1.30 ± 0.20 | — | −1.09 ± 0.09 | −0.22 ± 0.05 | — |
| 7 | 1.48 ± 0.20 | 0.80 ± 0.15 | −1.15 ± 0.09 | −0.24 ± 0.04 | — |
| 8 | 1.38 ± 0.20 | 0.99 ± 0.16 | −1.12 ± 0.09 | −0.29 ± 0.05 | — |

| Model | Magnesium | Phosphorus | Methionine | Cystine | Intercept | R2 |
|---|---|---|---|---|---|---|
| 1 | — | — | −0.32 ± 0.05 | — | 6.84 ± 0.07 | 0.08 |
| 2 | — | — | −0.30 ± 0.05 | — | 7.02 ± 0.07 | 0.13 |
| 3 | — | −0.47 ± 0.13 | −0.35 ± 0.05 | — | 7.50 ± 0.15 | 0.15 |
| 4 | — | −0.63 ± 0.14 | −0.38 ± 0.05 | — | 7.85 ± 0.17 | 0.18 |
| 5 | 6.10 ± 0.73 | −1.03 ± 0.14 | −0.29 ± 0.05 | — | 8.01 ± 0.16 | 0.28 |
| 6 | 4.85 ± 0.73 | −0.84 ± 0.14 | −0.24 ± 0.05 | — | 7.62 ± 0.17 | 0.34 |
| 7 | 4.59 ± 0.71 | −0.89 ± 0.13 | −0.33 ± 0.05 | — | 7.10 ± 0.19 | 0.37 |
| 8 | 4.51 ± 0.70 | −0.99 ± 0.13 | −0.45 ± 0.06 | 0.50 ± 0.16 | 7.02 ± 0.19 | 0.39 |

### Example 3

The cations included in the dry model were sodium, potassium, magnesium and calcium, whereas the anions were chloride, sulfur and phosphorus. Methionine and cystine were excluded from this model. The results are shown in Table 5. The analysis resulted in a prediction equation for dry foods. The new model accounted for 51% variation in individual (n=820) observed urine pH and 74% of the variation observed in average (n=82) urine pH in the cats fed 82 dry foods.

TABLE 5
Urine pH Prediction Models Determined by Stepwise Regression for Individual
Cats (n = 820) Fed 82 Dry Foods Using the Nutrient Components of the
Food (% Dry Matter Basis).

| Model | Sodium | Potassium | Chloride | Sulfur | Calcium |
|---|---|---|---|---|---|
| 1 | — | — | −1.10 ± 0.07 | — | — |
| 2 | — | 0.46 ± 0.06 | −1.11 ± 0.07 | — | — |
| 3 | — | 0.94 ± 0.06 | −0.90 ± 0.06 | −0.98 ± 0.08 | — |
| 4 | 1.21 ± 0.14 | 1.17 ± 0.07 | −1.12 ± 0.07 | −1.28 ± 0.08 | — |
| 5 | 0.98 ± 0.15 | 1.13 ± 0.07 | −1.14 ± 0.06 | −1.35 ± 0.08 | 0.39 ± 0.08 |
| 6 | 1.20 ± 0.15 | 1.19 ± 0.07 | −1.03 ± 0.06 | −1.51 ± 0.08 | 0.85 ± 0.10 |
| 7 | 1.03 ± 0.16 | 0.98 ± 0.09 | −0.83 ± 0.08 | −1.70 ± 0.10 | 0.85 ± 0.10 |

| Model | Magnesium | Phosphorus | Methionine | Cystine | Intercept | R2 |
|---|---|---|---|---|---|---|
| 1 | — | — | — | — | 7.32 ± 0.06 | 0.24 |
| 2 | — | — | — | — | 6.96 ± 0.07 | 0.30 |
| 3 | — | — | — | — | 7.11 ± 0.07 | 0.41 |
| 4 | — | — | — | — | 6.89 ± 0.07 | 0.46 |
| 5 | — | — | — | — | 6.72 ± 0.08 | 0.47 |
| 6 | — | −1.09 ± 0.16 | — | — | 7.01 ± 0.09 | 0.50 |
| 7 | 2.07 ± 0.57 | −1.15 ± 0.16 | — | — | 7.10 ± 0.09 | 0.51 |

### Results

The results from the stepwise regression analysis show which cations, anions and sulfur containing amino acids were of importance for urine pH prediction. Three models were developed for urine pH prediction. These models included foods, wet foods, and dry foods. The cations included in all models were sodium, potassium and magnesium. Calcium was included in the wet and dry and dry food model only. The anions for all models were chloride, sulfur and phosphorus. Including sulfur in the model allowed for the exclusion of methionine and cysteine from the dry model. The results show that urine pH can be predicted from the nutrient components of the food thus reducing the number of animal studies in order to optimize urine pH (for struvite and/or oxalate prevention) for specific products. Separate formulas can be used for dry and wet foods to maintain accuracy.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for predicting urine pH for an animal comprising:
determining the amount of selected cations, anions, and sulfur-containing amino acids in a food for consumption by the animal; and
predicting urine pH using a formula that equates the amount of such cations, anions, and sulfur-containing amino acids to urine pH.

2. The method of claim 1 wherein the cations are sodium, potassium, calcium, and magnesium; the anions are chloride, sulfur, and phosphorus; and the sulfur-containing amino acids are methionine and cystine; and the formula is (Formula 1):
Urine pH=AA+(AB*sodium)+(AC*potassium)−(AD*chloride)−(AE*sulfur)+(AF*calcium)+(AG*magnesium)−(AH*phosphorus)−(AI*methionine)−(AJ*cystine);
where AA is from about 5.5 to about 7.5; AB is from about 1.0 to about 1.3; AC is from bout 0.6 to about 0.9; AD is from about 0.6 to about 0.9; AE is from about 0.4 to about 0.9; AF is from about 0.8 to about 0.3; AG is from about 1.0 to about 1.5; AH is from about 0.5 to about 1.0; AI is from about 0.1 to about 0.5; and AJ is from about 0.1 to about 0.5.

3. The method of claim 1 wherein the cations are sodium, potassium, and magnesium; the anions are chloride, sulfur, and phosphorus; and the sulfur-containing amino acids are methionine and cystine; the food is a wet food; and the formula is (Formula 2):
Urine pH=WA+(WB*sodium)+(WC*potassium)−(WD*chloride)−(WE*sulfur)+(WG*magnesium)−(WH*phosphorus)−(WI*methionine)−(WJ*cystine);
where WA is from about 5.5 to about 7.5; WB is from about 1.2 to about 1.5; WC is from about 0.8 to about 1.2; WD is from about 1.0 to about 1.3; WE is from about 0.1 to about 0.9; WG is from about 3.5 to about 5.5; WH is from about 0.7 to about 1.3; WI is from about 0.3 to about 0.7; and WJ is from about 0.3 to about 0.8.

4. The method of claim 1 wherein the cations are sodium, potassium, calcium, and magnesium; the anions are chloride, sulfur, and phosphorus; the food is a dry food; and the formula is (Formula 3):
Urine pH=DA+(DB*sodium)+(DC*potassium)−(DD*chloride)−(DE*sulfur)+(DF*calcium)+(DG*magnesium)−(DH*phosphorus);
where DA is from about 5.5 to about 7.5; DB is from about 1.0 to about 1.3; DC is from about 0.6 to about 0.9; DD is from about 0.6 to about 0.9; DE is from about 0.4 to about 0.6; DF is from about 0.8 to about 0.3; DG is from about 1.0 to about 1.5; and DH is from about 0.5 to about 1.0.

5. A device suitable for predicting urine pH for an animal comprising a means for predicting urine pH that uses a method comprising determining the amount of selected cations, anions, and sulfur-containing amino acids in a food for consumption by the animal and predicting urine pH using a formula that equates the amount of such cations, anions, and sulfur-containing amino acids to urine pH.

6. The device of claim 5 wherein the means accepts input from a user comprising the amount of sodium, potassium, calcium, magnesium, chloride, sulfur, phosphorus, methionine, and cystine in a food for consumption by an animal and utilizes all or a subset of the input and one or more of Formula 1, Formula 2, and Formula 3 to predict urine pH in the animal that consumes the food.

7. The device of claim 6 wherein the means is a website, software program, calculator, or computer.

8. A method for preventing or treating urolithiasis comprising:
determining a desirable urine pH range for an animal;
choosing a food containing selected cations, anions, and sulfur-containing amino acids;
predicting urine pH using a formula that equates the amount of such cations, anions, and sulfur-containing amino acids in the food to urine pH in the animal that consumes the food; and
feeding an urolithiasis preventing or treating amount of the food to the animal if the predicted urine pH is in the desirable pH range.

9. The method of claim 8 wherein the pH range is from about 5.5 to 6.5; the cations, anions, and sulfur-containing amino acids are all or a subset of sodium, potassium, calcium, magnesium, chloride, sulfur, phosphorus, methionine, and cystine; and the formula is Formula 1, Formula 2, or Formula 3 as appropriate for the chosen food and the selected cations, anions, and sulfur-containing amino acids.

10. The method of claim 8 further comprising administering the composition to the animal in conjunction with one or more urolithiasis agents.

11. A kit suitable for predicting urine pH for an animal and/or for preventing or treating urolithiasis comprising in separate containers in a single package or in a virtual package, as appropriate for the kit component, a device of claim 5 and one or more of (1) a food suitable for animal consumption, (2) an urolithiasis agent, (3) a urine pH diagnostic device, (4) a means for communicating information about or instructions for using urine pH diagnostic devices, (5) a means for communicating information about or instructions for adjusting or controlling urine pH, (6) a means for communicating information about or instructions for using the methods, devices, and kits of the present invention to predict urine pH, and (7) a means for communicating information about or instructions for preventing or treating urolithiasis.

12. A means for communicating information about or instructions for (1) using the methods, devices, and kits of the present invention to predict urine pH, (2) using the methods, devices, and kits of the present invention to prevent or treat urolithiasis, (3) using urine pH diagnostic devices in the present invention comprising a document, digital storage media, optical storage media, audio presentation, or visual display containing the information or instructions.

13. The means for communicating of claim 13 selected from the group consisting of a displayed website, brochure, product label, package insert, advertisement, and visual display.
